(19) United States
(12) Patent Application Publication
(10) Pub. No.: US 2006/0233726 A1
(43) Pub. Date: Oct. 19, 2006

(54) SPF scalp sun screen protector

(76) Inventor: Sam (D.C.) Nia, Cumming, GA (US)

Correspondence Address: R & G CONSULTANTS, LLC, 1927 PIERCE WAY, BUFORD, GA, 30519, US

(21) Appl. No.: 11/107,269

(22) Filed: Apr. 18, 2005

(51) Int. Cl. A61K 8/64; A61K 8/49

(52) U.S. Cl. 424/59; 424/70.9

(57) ABSTRACT

The SPF Scalp Sun Screen Protector is the only product that will protect the scalp and the hair of an individual with fine or thinning hair from the ultra violet rays of the sun. It is a lotion that an individual can use on his scalp for outdoor events, such as sports, working outside, or outside leisure activities. The main characteristic of this product is that the chemicals and compounds are uniquely combined together to create a product with the SPF Sun Screen Protector that, in using the lotion and drying the hair, it can also be used to style the hair since it has some styling gel ingredients incorporated into it. The only Scalp Sun Screen Protector with Biotin Binding Proteins for enhancing the health and appearance of the hair.

### PURPOSE AND USE

The purpose of the SPF Scalp Sun Screen Protector is to protect an individual' scalp who has fine hair, or hair that has thinned out, from the ultra violet rays of the sun. It can be used by balding individuals, but it is not necessarily for these individuals since there are sun screen products for skin without hair. When an individual is out in the sun playing a sport, working around the yard, or just enjoying the sun, it is very likely that they will get sunburn or possible injury from the sun. Most individuals with thinning or fine hair will often develop a headache from the ultra violet rays. These headaches, along with the damage to the scalp over a period of time, can result in skin blisters or even skin cancer. The purpose of the SPF Scalp Sun Screen Protector is also to have a gel product to protect the scalp that, when applied, will dissolve for styling purposes and at the same time will have an SPF factor that serves as a sun screen against the ultra violet sun rays.

In using the SPF Scalp Sun Screen Protector, it is important that the individual applies the SPF Scalp Sun Screen Protector in moderation. What is important is that all surfaces of the hair area are covered with the Protector. An abundance of the Protector will cause the hair to feel heavy and sticky and will not create anymore protection than a moderate layer on the scalp. The solution can then be dried if the user chooses to do so. The drying process allows the Protector to act as a styling agent for the user and in the dried stage will also protect the scalp against the environment.

Chemical Makeup of the SPF Scalp Sun Screen Protector

The chemicals and compounds listed in FIG. 1 and FIG. 2 were put together to create a product that would cover the concept presented here by the inventor. This product was created from a formula for SPF skin protection and a formula from a gel that is used for hair that will dissolve when applied to the hair. When comparing the two formulas required for the Scalp Sun Screen Protector, and taking out the chemicals or compounds that were the similar in both and served the same purpose, a formula was derived for the SPF Scalp Sun Screen Protector. An ingredient know as Biotin Binding Protein has also been added to enhance the hair follicles. The analysis was done by using analysis procedures from Chemical Corp, which is an internet domain that can compare chemicals and their uses for chemical analysis. The reason for this is that combining it with the gel and applying it to the hair will cause the SPF factor to be lowered. It is estimated that this combination of the (2) ingredients (FIGS. 1 & 2) gave the product an SPF rating of approx. 15. FIGS. 3 and 4 show the ingredients required to increase the SPF rating to SPF 30 or SPF 45. As noted, many of the ingredients are the same except that FIG. 4 shows stronger and extra active ingredients (Avobenzone 2.0% w/w) to help achieve the SPF 45.

Many of the sun screen products on the market contain many of these same ingredients for hair styling products as for sun screen products. What the inventor has done is to combine these ingredients to create a product that could be used in the hair on a sunny day to protect the scalp. The gel ingredients remain the same for any variation of the SPF factor that the inventor chooses to create, from SPF 15 to SPF 45. A comparison of this is shown in (FIGS. 3 & 4), where the ingredients for SPF 30 and the ingredients for SPF 45 are shown. The inventor does not claim to have invented the ingredients or even the combination that makes up a hair gel or a sun screen, but has researched the combination of the ingredients to create a new product and concept called SPF Scalp Sun Screen Protector for the sole Purpose of Protecting the scalp on individuals with fine or thinning hair.

### DESCRIPTION OF THE FIGURES

FIG. 1: Shows the ingredients for the part of the SPF Scalp Sun Screen Protector that creates the gel base for dissolving into the scalp.

FIG. 2: Shows the ingredients for an SPF product that, when combined with the gel ingredients in FIG. 1, would create a product with an SPF factor of 15.

FIG. 3: Shows the ingredients for an SPF product that, when combining with the gel ingredients in FIG. 1, would create a product with an SPF factor of 30.

FIG. 4: Shows the ingredients for an SPF product that, when combining with the gel ingredients in FIG. 1, would create a product with an SPF factor of 45.

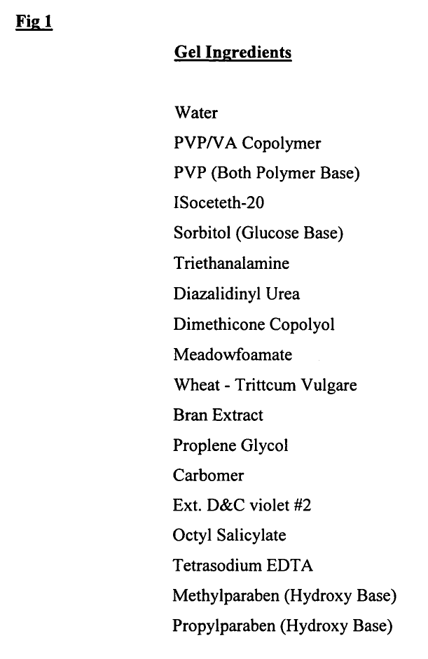

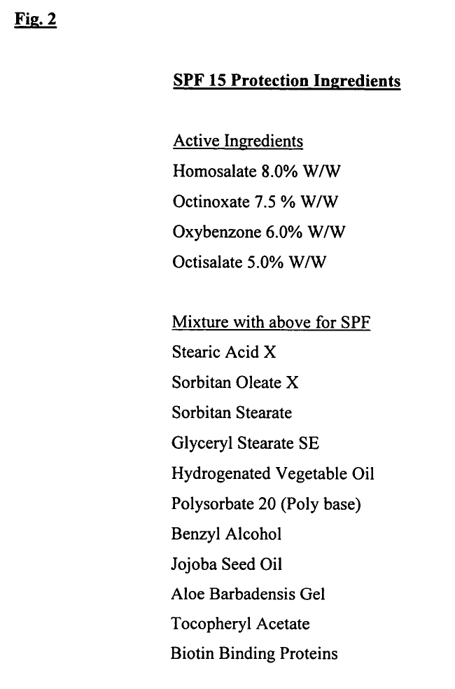

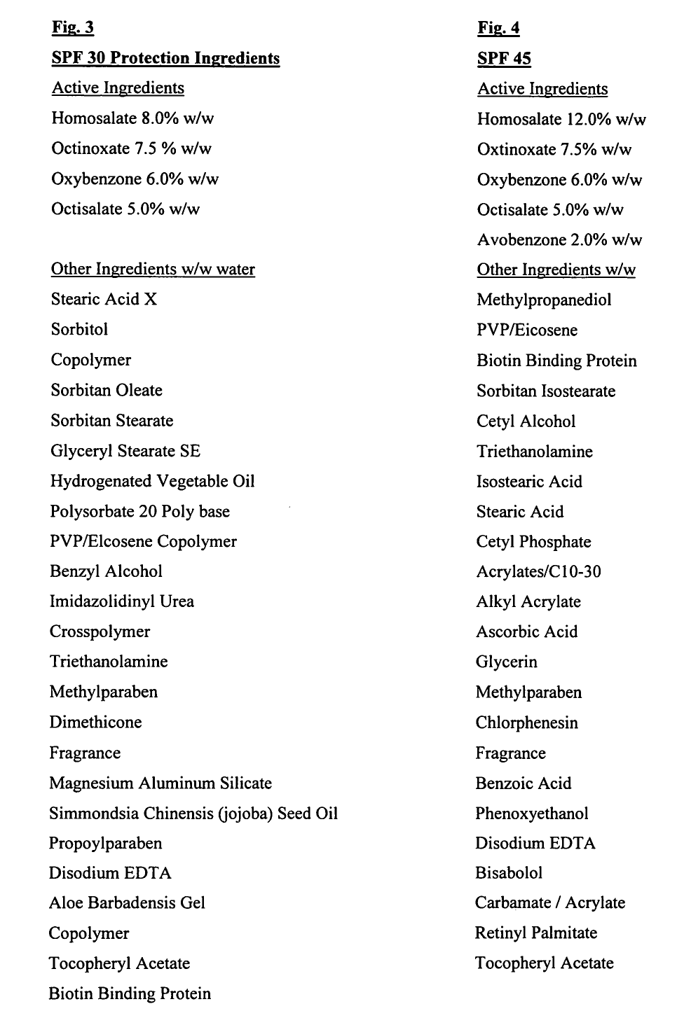

I claim:

1. I claim that the SPF Scalp Sun Screen Protector is the only sun screen that protects the scalp, as well as the hair, from sun damage on either a hot sunny day or an overcast day.

2. I claim that the SPF Scalp Sun Screen Protector is the only sun screen protector that has Biotin Binding Protein that will enhance the hair follicles, giving the hair more body.

3. I claim that the SPF Scalp Sun Screen Protector is the only sun screen protector that can be manufactured with different SPF factors to protect the scalp with variations of thinning hair.
